(19) United States
(12) Patent Application Publication
(10) Pub. No.: US 2014/0248322 A1
(43) Pub. Date: Sep. 4, 2014

(54) DENTAL COMPOSITIONS CONTAINING SILICA MICROBEADS

(71) Applicant: Robert L. Karlinsey, Indianapolis, IN (US)

(72) Inventor: Robert L. Karlinsey, Indianapolis, IN (US)

(21) Appl. No.: 14/278,447

(22) Filed: May 15, 2014

Related U.S. Application Data
(63) Continuation-in-part of application No. 14/176,584, filed on Feb. 10, 2014.
Application No. 14/176,584 is a continuation-in-part of application No. 13/438,318, filed on Apr. 3, 2012.
Application No. 13/438,318 is a continuation-in-part of application No. 13/860,007, filed on Apr. 10, 2013.
Application No. 13/860,007 is a continuation-in-part of application No. 13/438,318, filed on Apr. 3, 2012.
(60) Provisional application No. 61/471,649, filed on Apr. 4, 2011.

(51) Int. Cl. A61K 8/02; A61Q 11/00; A61K 8/25

(52) U.S. Cl. CPC A61K 8/025; A61K 8/25; A61Q 11/00
USPC 424/401; 424/57; 424/52

(57) ABSTRACT

A dental treatment system for the relief of dental hypersensitivity and related disorders, including a dentition delivery formulation and a plurality of generally spherical vitreous microspheres generally homogeneously dispersed in the dentition delivery formulation, wherein each respective microsphere has a coating containing calcium and phosphate and wherein each respective microsphere is between about 0.01 micron and about 10 microns in diameter.

### CROSS-REFERENCE TO RELATED APPLICATIONS

This patent application claims priority to co-pending U.S. patent application Ser. No. 14/176,584, filed on Feb. 10, 2014, which claimed priority to co-pending U.S. patent application Ser. No. 13/438,318, filed on Apr. 3, 2012, which claimed priority to then co-pending U.S. Provisional Patent Application Ser. No. 61/471,649, filed on Apr. 4, 2011; and to co-pending U.S. patent application Ser. No. 13/860,007, filed on Apr. 10, 2013, which claimed priority to co-pending U.S. patent application Ser. No. 13/438,318, filed on Apr. 3, 2012, which claimed priority to then co-pending and now abandoned U.S. Provisional Patent Application Ser. No. 61/471,649, filed on Apr. 4, 2011.

### BACKGROUND

Dental hypersensitivity is a challenging problem since the oral environment is continuously cycled through periods of demineralization (i.e. loss of mineral through acid attack or physical attrition) and remineralization (i.e. seeding minerals such as fluoride, calcium and phosphate that combine with the existing tooth structure to grow new mineral that can strengthen the enamel and/or dentin). When too much mineral is lost and the remineralization processes cannot keep the pace of replacing the lost mineral, tooth sensitivities ultimately develop. This is further complicated due to natural aging of the tooth and tends to be more common when restorative or implant procedures have been performed. Such sensitivity is typically manifested in pain arising from sudden extreme temperatures (such as drinking ice cold or steamy hot beverages) or changes in pressure, including the act of chewing or biting on brittle surfaces or through probing with a dental explorer or pressurized air. The sensitivities develop due to the exposure of nerves positioned within the dentin component of the tooth structure. Over time, the penetration of acids into and/or the thinning of enamel increases the risk of demineralizing the thin mineral layers in dentin that surround and protect the sensitive nerve endings. These nerves are typically positioned in dentin tubules (about 1-3 μm in diameter and at least 5 μm in length). Without adequate acid-resistant support, these nerves become triggered during an extreme event, such as chewing food, eating ice cream or drinking a hot beverage. Based on various surveys and polls, at least 40% of the population exhibits some dental hypersensitivity. Thus, hypersensitivity remains a challenging problem and opportunity.

There are several treatments currently used to treat hypersensitivity. One treatment is the placement of resins or varnishes on the affected area. This is typically performed by the dental professional, which may require frequent dental visits. Other treatments may include treating with higher levels of fluoride, such as 5,000 ppm fluoride toothpaste available through the dentist, or using a multiple agent product, such as toothpastes containing combinations of calcium, silica, fluoride, phosphate, strontium, and the like. The most common over-the-counter approach typically involves toothpastes containing potassium nitrate: although a barrier is not formed, the nitrate responds to and neutralizes the exposed nerve ending. These approaches have all produces significant benefits, however, problems still occur. For instance, some have aversions to high fluoride products while others may not visit the dentist on a regular basis. Additionally, the resin and potassium nitrate approaches are temporary solutions, requiring continuous use in order to enjoy long-term relief from hypersensitivity. Separately, the mineral formations that develop in and on the dentin through use of a multiple agent combination product may, over time, not provide sufficient protection against acid challenges and/or physical attrition. Therefore, in this disclosure, we describe a novel combination of materials for improved relief from dental hypersensitivity that also avoids the weaknesses associated with these existing therapies.

### DESCRIPTION OF THE DRAWINGS

FIG. 1 is a perspective view of a functionalized microbead according to a first embodiment of the present novel technology.

FIG. 2A is a schematic view of a tooth.

FIG. 2B is an enlarged portion of the tooth of FIG. 2A, illustrating the enamel outer surface.

FIG. 2C is an enlarged portion of the enamel outer surface of 2B, illustrating the tubules.

FIG. 3A is an enlarged view of the tubules of FIG. 2C showing a tubule.

FIG. 3B is a schematic view of the tubule of FIG. 3A occluded with an agglomeration of the microbeads of FIG. 1.

FIG. 4 illustrates a plan view of a microbead composition according to a second embodiment of the present novel technology.

FIG. 5A is an enlarged view of dental tubules.

FIG. 5B is a schematic view of the tubule of FIG. 3A occluded with the composition of FIG. 4.

FIG. 5C is a schematic view of the tubule of FIG. 3A occluded with the composition of FIG. 4 after hardening into an agglomeration.

FIG. 6 is a 30,000× photomicrograph of silica microspheres coated with a calcium and phosphate containing layer according to a third embodiment of the present novel technology.

FIG. 7 is a 60,000× photomicrograph of the embodiment of FIG. 6.

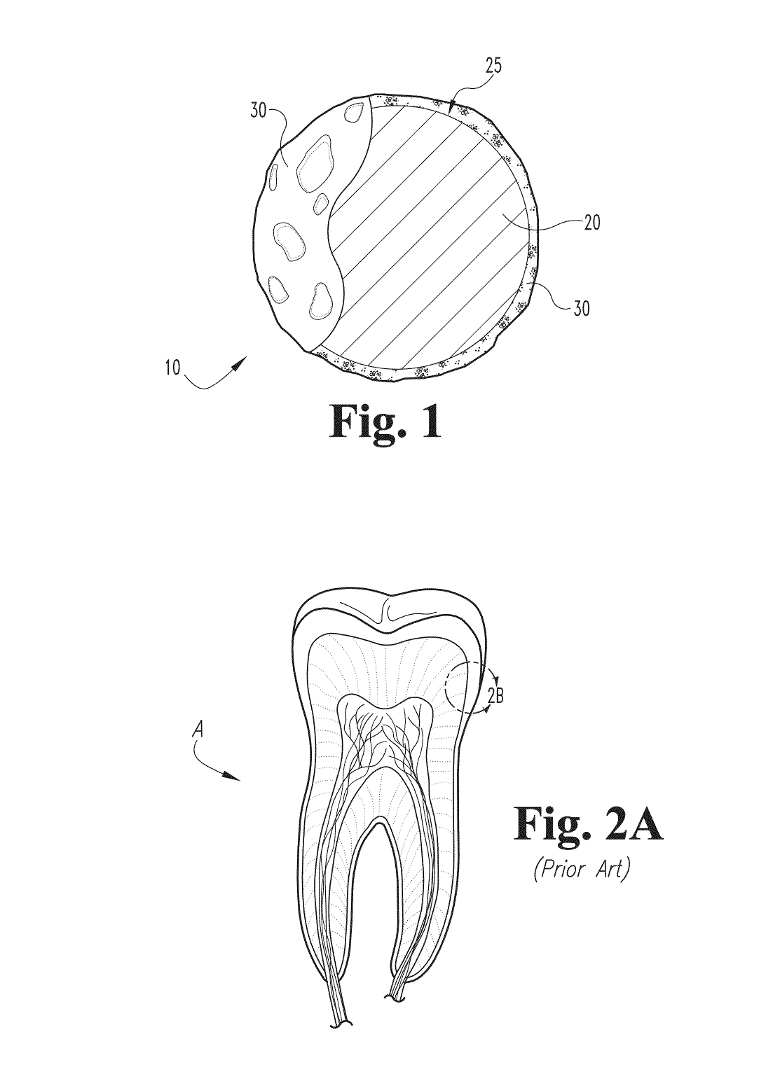

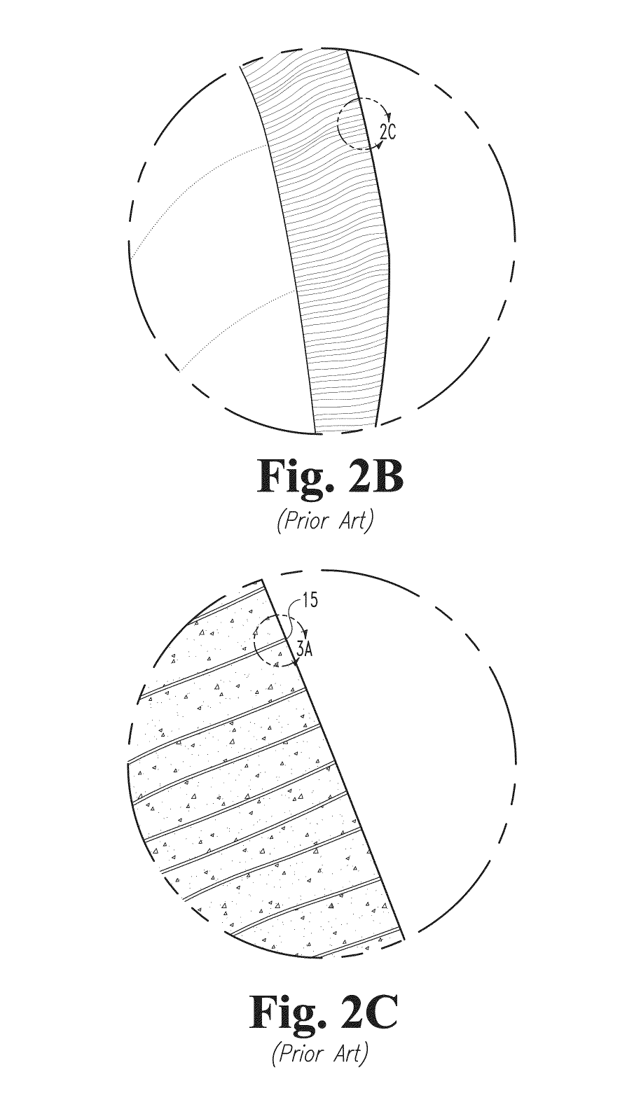

### DETAILED DESCRIPTION

For the purposes of promoting an understanding of the principles of the novel technology, reference will now be made to the embodiments illustrated in the drawings and specific language will be used to describe the same. It will nevertheless be understood that no limitation of the scope of the novel technology is thereby intended, such alterations and further modifications in the illustrated device, and such further applications of the principles of the novel technology as illustrated therein being contemplated as would normally occur to one skilled in the art to which the novel technology relates.

The present technology relates to the general reduction in dental hypersensitivity. One feature of this technology is the incorporation of microbeads 10 into exposed dentin tubules 15. Another feature is the acid-resistant adhesion and retention of the microbeads 10 within the dentin tubules 15 as well as on the weakened dentin surfaces that has resulted from demineralization processes. The microbeads 10 typically include an inorganic microsphere 20 that is coated with a thin organic coating 30. This two-phase system 10 provides a physically strong, acid-resistant layer 30 that adheres to the demineralized dentin surfaces and also occludes dentin tubules 15. This two-phase system 10 can be implemented into dental vehicles including toothpastes, rinses, varnishes, gums, mints, gels, and the like.

Inorganic materials such as silica, titania, alumina, various glass compositions, and the like are commonly found in dental products and are used, for instance, as abrasives, fillers, pigments and/or for providing structural rigidity, and thus may also be used for the inorganic microsphere or core portion 20. Alternately, polymeric microspheres 20 such as polyethylene may also be used for the core portion 20. Microspherical shapes of these materials offer significant benefits relative to other geometries, including yielding large surface areas available for functionalization. Spherical or near-spherical geometry also provides a favorable shape for penetration into demineralized dentin tubules 15, which are porous in nature with diameters between 1 and 4 μm. The spherical shape can also affect light reflection to create a ‘whitening-like’ effect, both from the reflectivity of the round unfunctionalized silica surfaces, the reflectivity of various coatings, and/or the white color of microspheres composed of titania, alumina or the like.

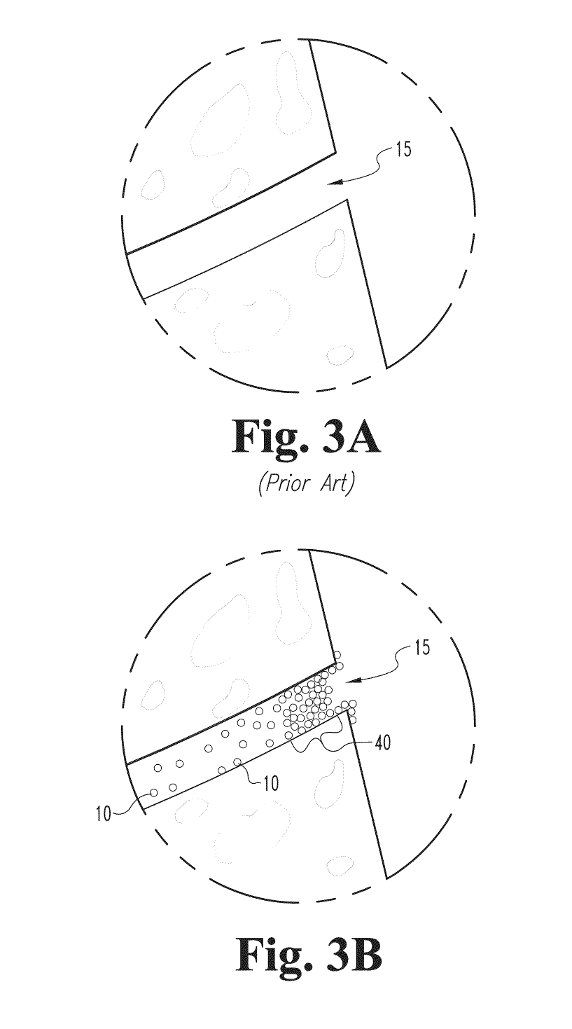

As shown through in microscopy analysis of demineralized dentin (see FIG. 3B), the microspheres 10 can readily penetrate into the dentin tubule. Due to differences in the physical and chemical properties between organic (e.g. polymers and the like) and inorganic (e.g., silica and the like) materials, inorganic materials such as silica, titania, and the like can provide structural rigidity and resistance to acid attack, which are attractive and beneficial features when embedded into the relatively softer and acid-susceptible dentin tubules 15.

The present novel technology relates to microbeads 10, typically having dimensions between 0.01 μm to 10 μm, and more typically between 0.9 μm and 3 μm to encourage optimized packing within the dentin tubules. However, other convenient dimensions may be selected. More typically, microbeads 20 are provided with a particle size distribution (PED) defining a variety of diameters within this range for optimal packing and thus the provision of hypersensitivity benefits. Although the microspheres 20 are typically composed of inorganic materials, such as silica, titania, and the like, organic microspheres 20 made from such materials as polyethylene, polypropylene, cross-linked polymers and the like may also be used.

Although the microspheres 20 can penetrate readily into the dentin tubule 15, they are less adept at attaching to the relatively flat dentin surfaces. Therefore, the microsphere surface 25 may include an adhesive coating 20 to encourage greater adhesion of the smooth spherical surface 25 to the smooth dentin surface. Once in the tubule 15, a plurality of microbeads 10 adhere to the tubule walls and each other defining an agglomerate 40 that effectively occludes the tubule 15.

One approach to providing an adhesive coating 30 is to coat or functionalize the inorganic sphere 20 with a hydrophilic or sticky material layer 30. This material 30 can be organic, including a polymer such as polyacrylic acid (e.g. typical molecular weight range between 100,000 and 450,000 g/mol), or another, typically hydrophilic, material. The coating material 30 may be phosphate, such as derived from phosphoric acid. Alternately, the coating material 30 may be hydrophobic, such as derived from silanes or methanes. Typically, the material 30 used to coat the microbeads 20 has some character that encourages mineral seeding and growth so that new mineral can be formed over time within the dentin tubule as well as on the dentin surface. This coating material 30 can attract ions available from both the natural oral environment through saliva components, including calcium, phosphate and the like, and also from the use of dental products containing, for example, fluoride, calcium, phosphate, strontium, potassium, nitrate, tin and the like.

In constructing the functionalized microbeads 10, the typical weight fraction of the organic material 30, such as phosphoric acid, polyacrylic acid and the like, may typically range between 0.01% and 5%, more typically between 0.1% and 1%. The corresponding weight fraction of the organic material 30 should be combined with a suspension or dry powder of inorganic microspheres 20, typically silica or titania. This combination can be achieved using typical chemical approaches including dehydration of a silica suspension followed by addition of the organic agent in the stated amounts coupled with a dehydration step to achieve a dry powder. Alternately, a suspension of the microspheres 20 can be combined with the stated amount of organic agent 30 and left as a suspension.

The two-phase combination 10, comprising microspheres 20 coated with an adhesive layer 30, are able to provide additional benefits relative to use of either agent alone. The adhesive property of the functionalized microsphere 10 can remain on the dentin surface despite challenge from an acid attack, providing structural stability and improved chemical resistance to demineralization from enamel and dentin. The coating 30 serves additional purposes: namely, a sacrificial layer 30 to subsequent acid attacks as well as to promote remineralization through contact with saliva and various dental products with or without fluoride. These factors bear directly on the relief from dentin demineralization and therefore dental hypersensitivity. While either the microbeads 10 alone or the organic coating material 30 alone could offer hypersensitivity relief, the two-phase composite bead 10 can extend hypersensitivity relief. Thus, these composite beads 10 may be implemented into mints, lozenges, gums, rinses, pastes, gels, and the like in form of dry powders or suspensions for delivery to tubules 15 and dentin upon introduction into the oral cavity.

One example of a treatment is the application of a 10% w/v suspension of 1 μm diameter silica microspheres 10 functionalized with 0.5% phosphoric acid coating 30 to demineralized dentin. Application of functionalized microbeads 10 to dentin/tubules may provide greater resistance to acid challenges compared to native (i.e. unfunctionalized) silica microspheres 20.

### DETAILED EXAMPLE

One method of functionalizing silica microspheres with phosphate (PO₄) was produced with phosphoric acid is as follows:
1) Using a vortex mixer, shake silica suspensions (formulated as discussed above) thoroughly for several minutes.
2) Extract 1 ml of the silica microsphere solution (number of microspheres 10 is approximately 10⁹, 10¹⁰, and 10¹¹ microspheres 10 for 1.0, 2.5, and 4.0 μm diameter microspheres, respectively) and place in a glass container (i.e. 50 ml Pyrex beaker).
3) Place the beaker with the 1 ml solution into the vacuum oven (warmed to ˜100° C.) and slowly pull a vacuum—let it stand for about five minutes or later until the water is removed. Only the silica 20 should remain.
4) To clean the silica 20 and prepare it for functionalization, add several milliliters of ethanol to the resultant powder and then evaporate it—place it in the 100° C. oven for ˜10 minutes to remove ethanol.
5) If desired, repeat Step #4 one more time.
6) Separately, make ˜0.5% w/w H₃PO₄(aq) (i.e. 500-fold dilution of 85% w/w parent solution using distilled water).
7) Add 2 ml of the acidic solution 30 to the silica powder 10 and gently mix—place in oven (e.g. ˜100° C. for 15 minutes and slowly pull a vacuum to encourage evaporation.
8) Collect the resultant acid-functionalized silica powder 10, weigh it, and set it aside in a sealed container for later use.
   Using PO₄ functionalized microspheres 10, bovine dentin was demineralized to expose the ˜2-5 μm diameter tubules (50% citric acid solution, ten minutes, room temperature), then treated with a small drop of a 10% suspension (30 mg into 0.3 ml distilled water). Observations were then obtained using scanning electron microscopy.
   Amount of Recovered Sample after PO₄ Procedure:

| Silica | |
|---|---|
| Microsphere Diameter | Mass (mg) |
| 1.0 μm | 110.1 |
| 2.5 μm | 114.6 |
| 4.0 μm | 110.6 |

Using PO₄ functionalized microspheres, bovine dentin was demineralized to expose the ˜2-5 μm diameter tubules (50% citric acid solution, 10 minutes, room temperature), then treated with a small drop of a 10% suspension (30 mg into 0.3 ml distilled water). Observations were then obtained using scanning electron microscopy.

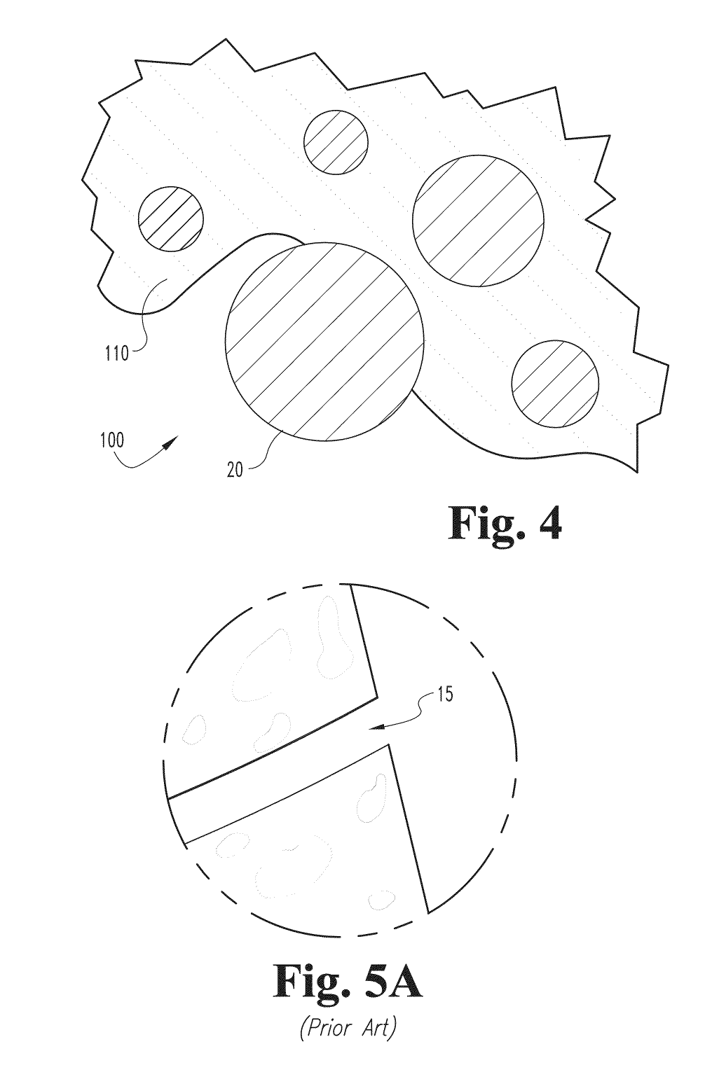

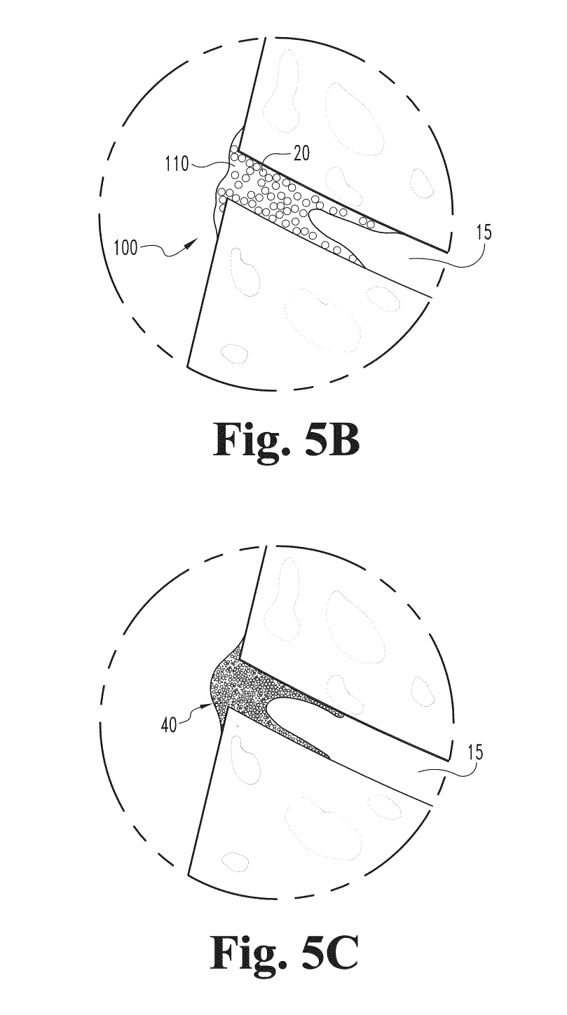

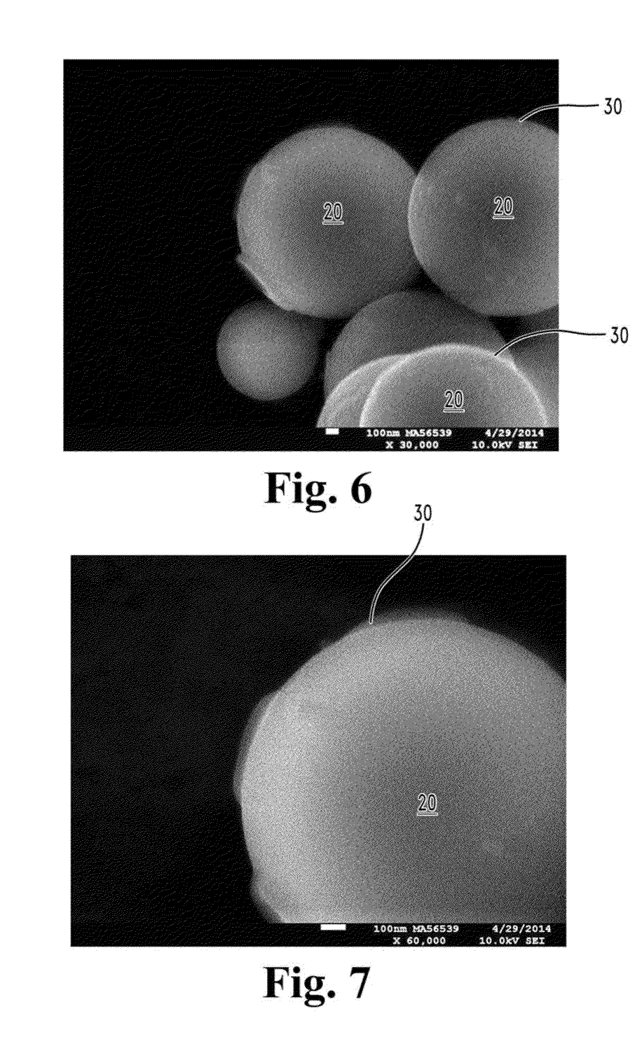

As illustrated in FIGS. 4 and 5A-5C, another embodiment of the present novel technology relates to a system 100 including a plurality of unfunctionalized or ‘naked’ microbeads 20, again typically having dimensions between 0.01 μm to 10 μm, and more typically between 0.9 μm and 3 μm, for packing within dentin tubules. More typically, microbeads 20 are provided with a particle size distribution (PED) defining a variety of diameters within this range for optimal packing and thus the provision of hypersensitivity benefits. Again, the microspheres 20 are typically composed of inorganic materials, such as silica, titania, glass (including bioresorbable glass such as 45S5 glass and like compositions), and/or organic materials such as polyethylene, polypropylene, cross-linked polymers and the like, or combinations thereof.

The microspheres 20 when introduced alone may penetrate readily into the dentin tubule 15, where they can agglomerate to form occlusions. Typically, the microspheres 20 are introduced with a dental delivery mechanism 110, such as a varnish, that both transports the microspheres 20 into the oral cavity to the dentition, and assists in adhering the microspheres 20 to the tubules 15. Once in the tubule 15, a plurality of microbeads 10 adhere to the tubule walls and to each other via the delivery fluid matrix 110 to define an agglomerate 40 that effectively occludes the tubule 15.

As with the functionalized microbead embodiment discussed above, this system 100 defined by unfunctionalized microbeads 20 suspended in a delivery fluid 110 defines a two-phase combination 100, comprising microspheres 20 suspended in a typically adhesive medium 110 for providing additional benefits relative to use of either agent alone. The unfunctionalized microsphere 20 may become adhered to a dentin surface via the delivery medium 110, such as varnish, despite challenge from an acid attack, providing structural stability and improved chemical resistance to demineralization from enamel and dentin. The varnish 110 likewise serves additional purposes, such as providing a sacrificial layer to subsequent acid attacks as well as to promote occlusion and/or remineralization through contact with saliva and various dental products with or without fluoride. These factors bear directly on the relief from dentin demineralization and therefore dental hypersensitivity. While either the microbeads 10 alone or the (typically organic) coating material 110 alone could offer hypersensitivity relief, the composite agglomerate 40 extends hypersensitivity relief.

### EXAMPLE

### Non-Functionalized Silica Microspheres

The second example is a variant of the first example, wherein increasing occlusion is achieved using non-functionalized silica microspheres 20. The silica microspheres 20 are less than 3 μm in diameter and provide structural rigidity and resistance to acid attack of dentin. Much like the functionalized microspheres, the non-functionalized silica microspheres 20 are compatible with dentin and are able to attach to the walls of the tubules 15 as well as each other via the carrier medium 110, to participate in occluding dentin and obstructing future acidic interactions or erosion. These naked microspheres 20 may be implemented into dental vehicles 110 including varnishes, dentifrices, mouthwash and the like and may include fluoride agents to assist in whitening and dentin remineralization. The non-functionalized silica microspheres 20 have been observed to form agglomerates 40 that occlude the dentin tubules 15 and resist acid attack. Dentin specimens were demineralized for 10 minutes using 50% citric acid (pH=1.59). The specimens were then treated with the non-functionalized silica microspheres 20 in a 10% w/v suspension, and then again exposed to a subsequent 1% citric acid (pH=3.8) for 5 minutes. The study showed the microspheres' 20 ability to penetrate and remain in tubules 15 during the subsequent citric acid attack.

In one embodiment, the delivery material 110 is a prophy composition, such as a prophy powder or prophy paste, typically containing a powdered abrasive material 125, such as glycine, sodium bicarbonate, calcium bicarbonate, calcium phosphate, or the like. The powdered abrasive material typically comprises at least about 80 volume percent of the prophy composition, more typically at least about 90 volume percent. The addition of a predetermined amount of microbeads 10, either functionalized or non-functionalized, enhances the prophy paste or powder 110 by contributing to both the polishing efficacy of the prophy powder 110 due to the inherent abrasive character of the microbeads 10 as well as providing a sensitivity decreasing character from the microbeads 10 ability to block or clog tubules 15. The presence of an organic coating 30 on functionalized microspheres 20 serves to decrease their contribution to polishing while increasing the efficacy of the functionalized microspheres 20 as tubule-blocking sensitivity reducing agents.

In some embodiments, the microbead coating 30 includes a chemical whitening agent, such as a bleaching agent or colorant, that may be imparted to reduce staining and whiten the dentition.

It will be appreciated that several of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following claims.

In one embodiment, the microspheres 20 include a coating layer 30 containing both calcium and phosphate, typically together as calcium phosphate. Typically, the calcium phosphate layer 30 is produced from the addition of calcium glycerophosphate, although other salts, such as calcium chloride, calcium sulfate, and the like may be combined concomitantly with phosphate salts, such as alkali metal phosphates including disodium hydrogen phosphate, potassium phosphate, sodium monofluorophosphate, and the like. The selected calcium and/or phosphate salts may be sparingly soluble, partially soluble or soluble in water.

The microspheres 20 are typically silica or silica-based glass, but may be any vitreous, biocompatible or nontoxic material, such as soda-lime-silica glass, borate glass, bioglass (such as 45S5 or other formulations), or the like. Alternately, the microspheres 20 may be ceramic metal oxides, such as alumina, titania, aluminosilicate, or the like.

Typically, calcium phosphate coatings may be accomplished under acidic, neutral or basic conditions. The coatings 30 may include an organic functionalization portion. The coatings 30 may be adhesive. The microspheres 20 including calcium and phosphate in their coating layers 30 may be introduced into such dentition delivery formulations or dental products as prophy pastes, varnishes, dentifrices, gels, creams, gum, mints, sprays, mouthwashes, whitening systems and the like. Such dental products may optionally include fluoride.

Table 1 illustrates x-ray photoelectron spectroscopy data collected from a plurality of calcium/phosphate coated 30 microspheres 20.

TABLE 1
XPS data collected using a 200-micron diameter x-ray beam.
The six different areas above pertain to different regions of
the sample, with each region containing multiple microspheres.
Carbon, oxygen, sodium, silicon, calcium and phosphorous
were all detected.

| | Atomic Percentage | | | | | |
|---|---|---|---|---|---|---|
| | | | | | Phos- | |
| Area | Carbon | Oxygen | Sodium | Silicon | phorous | Calcium |
| 1 | 8.9 | 61.8 | 1.7 | 21.7 | 3.2 | 2.7 |
| 2 | 4.0 | 66.4 | 0.6 | 25.1 | 2.3 | 1.7 |
| 3 | 5.3 | 65.5 | 1.7 | 23.8 | 2.1 | 1.7 |
| 4 | 6.9 | 62.8 | 2.7 | 22.7 | 2.9 | 2.0 |
| 5 | 6.5 | 65.2 | 2.0 | 20.7 | 3.0 | 2.6 |
| 6 | 8.7 | 61.2 | 2.7 | 21.7 | 3.1 | 2.5 |
| AVERAGE | 6.7 | 63.8 | 1.9 | 22.6 | 2.8 | 2.2 |
| SD | 1.9 | 2.2 | 0.8 | 1.6 | 0.5 | 0.5 |

While the invention has been illustrated and described in detail in the drawings and foregoing description, the same is to be considered as illustrative and not restrictive in character. It is understood that the embodiments have been shown and described in the foregoing specification in satisfaction of the best mode and enablement requirements. It is understood that one of ordinary skill in the art could readily make a nigh-infinite number of insubstantial changes and modifications to the above-described embodiments and that it would be impractical to attempt to describe all such embodiment variations in the present specification. Accordingly, it is understood that all changes and modifications that come within the spirit of the invention are desired to be protected.

I claim:

1. A composition for the relief of dental hypersensitivity and related disorders, comprising a plurality of generally spherical vitreous microspheres generally homogeneously dispersed in an oral delivery formulation; wherein the microspheres are selected from the group comprising silica, silicate glass, borate glass, bioglass, and combinations thereof; wherein each respective microsphere has a coating containing calcium and phosphate; and wherein each respective microsphere is between about 0.01 micron and about 10 microns in diameter.

2. The oral delivery system of claim 1 is a cleaning and polishing paste, including a dentifrice and prophylaxis paste.

3. The oral delivery system of claim 1 is a gel or varnish.

4. The oral delivery system of claim 1 is a mouthwash.

5. The oral delivery system of claim 1 is a gum or mint.

6. The oral delivery system of claim 1 contains fluoride.

7. The dental system of claim 1 wherein the respective coatings further comprise an organic functionalized portion.

8. The dental system of claim 3 wherein the organic functionalized portion is an adhesive.

9. A dental hypersensitivity treatment system, comprising:
a plurality of generally spherical microbeads;
a plurality of shells, each respective shell at least partially coating a respective generally spherical microbead; and
am oral delivery medium;
wherein each respective shell contains calcium and phosphate;
wherein the plurality of generally spherical inorganic microbeads are homogeneously suspended in the oral delivery medium to define a dental hypersensitivity treatment composition.

10. The dental treatment system of claim 9, wherein the generally spherical inorganic microbeads are generally composed of an inorganic material from the group comprising silica, titania, alumina, silicate glass, borate glass, bioglass and combinations thereof.

11. The dental treatment system of claim 9, wherein each respective generally spherical inorganic microbead is between 0.01 μm to 3 μm in diameter.

12. The dental treatment system of claim 9, wherein the delivery medium is selected from the group comprising prophy pastes, varnishes, dentifrices, gels, creams, gum, mints, sprays, mouthwashes, whitening systems, and combinations thereof.

13. The dental treatment system of claim 9, wherein the delivery system includes fluoride.

14. The dental treatment system of claim 9, wherein the delivery medium adheres to at least two generally spherical inorganic microbeads and to dentin surfaces to define a composite aggregate, and wherein the composite aggregate provides dental remineralization and a sacrificial layer for acid resistance.

15. The dental treatment system of claim 9, wherein the respective shells include a functionalized organic portion.

16. The dental treatment system of claim 15 wherein the organic portion is an adhesive.

17. The dental treatment system of claim 9 wherein the shells include calcium phosphate.
